(19) United States
(12) Patent Application Publication
(10) Pub. No.: US 2024/0245775 A1
(43) Pub. Date: Jul. 25, 2024

(54) Treatments to cancers /tumors also skin aligments /rushes

(71) Applicant: Menni Menashe Zinger, Ashkelon (IL)

(72) Inventor: Menni Menashe Zinger, Ashkelon (IL)

(21) Appl. No.: 18/100,560

(22) Filed: Jan. 23, 2023

(51) Int. Cl. A61K 45/06; A61P 3/10; A61P 17/00; A61P 35/00; A61P 37/08

(52) U.S. Cl. CPC A61K 45/06; A61P 3/10; A61P 17/00; A61P 35/00; A61P 37/08

(57) ABSTRACT

I present here new methods and theory about cancer and cancer treatment/curing also skin rushes.

### THEORY AND INVENTION

using injection that aimed to treat diabetes 2 and or as diet might cause cancer because of reducing the level of glucose that going into the cells and the body uses iron instead and the iron lead oxygen better and too much oxygen cause sleepy to oxygen and that cause cancer at glands, skin, lungs/breading organs and I suggest that the treatment would be anti allergic medications as anti histamine and others that are excepted as treatment against allergies also against skin rushes.

1. invention and theory at medicine and physiology: The skin is also breading as the lungs and the oxygen or other gas might be involved at cancer there also at leukemia there is oxygen involved and the red cells are leading oxygen and get damaged from it or allergic to oxygen also lungs and skin got allergic to oxygen and Lung cancer and leukemia and skin cancer, tumors are similar and opposite from stomach cancer or other cancers, leukemia is opposite of Hodgkin illness and using anti allergic medication might cure/treat those cancers, lungs/skin/leukemia and using anti histamines medications, anti allergic medications should be the solution to those cancers epipen also also against skin rushes.
